(19) United States
(12) Patent Application Publication
(10) Pub. No.: US 2026/0151195 A1
(43) Pub. Date: Jun. 4, 2026

(54) POSITIONING METHOD AND SYSTEM FOR SURGICAL ROBOT, SURGICAL ROBOT AND STORAGE MEDIUM

(71) Applicant: Precision Robotics (Hong Kong) Limited, Shatin, N.T. (HK)

(72) Inventors: Cheuk Ho Yuen, Shatin, N.T. (HK); Hyeun Woo Chung, Shatin, N.T. (HK)

(21) Appl. No.: 19/121,255

(22) PCT Filed: Nov. 10, 2023
(86) PCT No.: PCT/CN2023/130926

§ 371 Date: Apr. 15, 2025

(30) Foreign Application Priority Data
Nov. 15, 2022 (HK) 22022063883.1

(51) Int. Cl. A61B 34/30; A61B 34/20; A61B 90/00

(52) U.S. Cl. CPC A61B 34/30; A61B 34/20; A61B 90/06; A61B 2034/2059; A61B 2090/067

(57) ABSTRACT

The present application provides a positioning method, system for a surgical robot, a surgical robot and a storage medium. Present first adjustment data of the joint angle corresponding to each joint of a surgical robot is determined according to present first detected data of the joint bending angle and present first estimated data of the joint bending angle of each joint. An orientation parameter and a movement parameter of the tip of an operating arm is obtained according to the first adjustment data of the joint angle and the length of the operating arm. In this way, the adjustment data of the joint angle can be determined by combining the detected data and estimated data of the joint bending angle, and the detected data of the joint bending angle can be calibrated to ensure the accuracy of the positioning of the surgical robot and the movement of the operating arm.

### CROSS-REFERENCE TO RELATED APPLICATIONS

This Application is a National Stage Application under 35 U.S.C. § 371 of PCT Application No. PCT/CN2023/130926, filed Nov. 10, 2023, which claims priority to Hong Kong patent application Ser. No. 22022063883.1, filed Nov. 15, 2022, the entire contents of which are incorporated by reference herein.

### TECHNICAL FIELD

The present application relates to the technical field of medical devices, in particular to a positioning method and system for a surgical robot, a surgical robot and a storage medium.

### BACKGROUND

The hinged surgical robot uses a mechanical arm to imitate the action of the human arm, and enables the mechanical arm to move flexibly through the configuration of rotatable joints. The surgical robot in the prior art would use strain gauges to sense the bending stress of joints, to position the mechanical arm, and feed back to the control system to control the next operation of the mechanical arm. However, in practical applications, the detected data detected by the strain gauges will be affected by temperature and other noises, which will lead to inaccuracy of the detected data of the strain gauges, and thus lead to inaccurate positioning of the mechanical arm, which affects the accuracy of the movement of the robotic arm.

### SUMMARY

In order to solve or at least partially solve the above technical problems, the present application provides a positioning method for a surgical robot, which is applied to a surgical robot. The surgical robot includes an operating arm, the operating arm includes a plurality of joint modules connected with each other, and a strain gauge is provide at each joint connected between the joint modules respectively.

The method includes:
obtaining a joint bending angle of each joint in the present (current) detection, and getting first detected data of the joint bending angle corresponding to each joint, where the joint bending angle is determined according to a resistance parameter detected by the strain gauge corresponding to the joint;
getting present first estimated data of the joint bending angle of each joint, according to previous second adjustment data of the joint angle of each joint;
determining present first adjustment data of the joint angle corresponding to each joint, according to the present first detected data of the joint bending angle and the present first estimated data of the joint bending angle of each joint; and
obtaining an orientation parameter and a movement parameter of a tip of the operating arm, according to the first adjustment data of the joint angle corresponding to each joint and a length of the operating arm.

Optionally, at least two strain gauges are installed on each joint, the obtaining the joint bending angle of each joint in the present detection, and getting the first detected data of the joint bending angle corresponding to each joint, including:
obtaining the resistance parameter of each strain gauge corresponding to each joint, and getting first resistance parameter data; and
determining a joint bending yaw angle and a joint bending pitch angle corresponding to each joint according to the first resistance parameter data, and taking the joint bending yaw angle and the joint bending pitch angle corresponding to each joint as the first detected data of the joint bending angle corresponding to the joint.

Optionally, the surgical robot further includes a motor, which is used to drive the operating arm, the getting the present first estimated data of the joint bending angle of each joint, according to the previous second adjustment data of the joint angle of each joint, including:
obtaining motor input angle data of the motor; and
determining the first estimated data of the joint bending angle corresponding to each joint according to the motor input angle data, the previous second adjustment data of the joint angle corresponding to each joint and a preset calibration parameter, and getting the present first estimated data of the joint bending angle corresponding to each joint, where the preset calibration parameter comprises a kinematic parameter and a motor shaft parameter.

Optionally, the determining the present first adjustment data of the joint angle corresponding to each joint, according to the present first detected data of the joint bending angle and the present first estimated data of the joint bending angle of each joint, including:
calculating bending angle covariance according to the present first estimated data of the joint bending angle of each joint;
obtaining measured noise covariance of the strain gauge;
determining the Kalman gain according to the bending angle covariance, the measured noise covariance and a preset strain gauge parameter; and
determining the first adjustment data of the joint angle corresponding to each joint according to the present first estimated data of the joint bending angle, the present first detected data of the joint bending angle and the Kalman gain of each joint, and getting the first adjustment data of the joint angle corresponding to each joint.

Optionally, the obtaining an orientation parameter and a movement parameter of a tip of the operating arm, according to the first adjustment data of the joint angle corresponding to each joint and a length of the operating arm, including:
determining a spatial transformation matrix of each pairwise adjacent joint modules from the joint module at a position of the tip to the distal joint module until the proximal joint module according to the first adjustment data of the joint angle corresponding to each joint and the length of the operating arm, and getting the spatial transformation matrix corresponding to each joint; and
determining the orientation parameter and the movement parameter of the tip according to the spatial transformation matrix corresponding to each joint.

The embodiments of the present application further provide a positioning system for a surgical robot.

The positioning system for a surgical robot is applied to a surgical robot and includes an operating arm. The operation arm includes a plurality of joint modules connected with each other, and a strain gauge is provide at each joint connected between the joint modules respectively. The operation arm is connected with a central processing unit, and the central processing unit is configured to:
obtain a joint bending angle of each joint in the present detection, and get first detected data of the joint bending angle corresponding to each joint, where the joint bending angle is determined according to a resistance parameter detected by the strain gauge corresponding to the joint;
get present first estimated data of the joint bending angle of each joint, according to previous second adjustment data of the joint angle of each joint;
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

The embodiments of the present application further provide a surgical robot.

The surgical robot comprises an operating arm. The operation arm includes a plurality of joint modules connected with each other, and a strain gauge is provide at each joint connected between the joint modules respectively. The operation arm is connected with a central processing unit, and the central processing unit is configured to:
obtain a joint bending angle of each joint in the present detection, and get first detected data of the joint bending angle corresponding to each joint, where the joint bending angle is determined according to a resistance parameter detected by the strain gauge corresponding to the joint;
get present first estimated data of the joint bending angle of each joint, according to previous second adjustment data of the joint angle of each joint;
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

The embodiments of the application further provide a computer-readable storage medium, which stores program instructions. When the program instructions are performed by a computer, cause the computer to perform the positioning method for a surgical robot as described above.

The embodiments of the application further provide a computer program product, where the computer program product includes a non-transitory computer-readable storage medium that stores computer programs, and the computer programs are operable to enable the computer to perform some or all of the steps described in any positioning method of a surgical robot recorded in the embodiments of the present application. The computer program product can be a software installation package.

In the embodiments of the application, the first detected data of the joint bending angle corresponding to each joint is got by obtaining the joint bending angle of each joint in the present detection. The joint bending angle is determined according to the resistance parameter detected by the strain gauge corresponding to the joint. The present first estimated data of the joint bending angle of each joint is got according to the previous second adjustment data of the joint angle of each joint. The present first adjustment data of the joint angle corresponding to each joint is determined according to the present first detected data of the joint bending angle and the present first estimated data of the joint bending angle of each joint. The orientation parameter and the movement parameter of the tip of the operating arm is obtained according to the first adjustment data of the joint angle corresponding to each joint and the length of the operating arm. In this way, the adjustment data of the joint angle can be determined by combining the detected data and estimated data of the joint bending angle, and the detected data of the joint bending angle can be calibrated to reduce the impact of noise such as temperature on the detection results, so as to ensure the accuracy of positioning the position and direction of the tip of the operating arm.

### BRIEF DESCRIPTION OF THE DRAWINGS

In order to more clearly explain the embodiments of the present application, the following will briefly introduce the relevant accompanying drawings. It can be understood that the accompanying drawings in the following description are only used to illustrate some embodiments of the present application, and general technicians in the art can also obtain many other technical features and connection relationships not mentioned herein according to these accompanying drawings.

FIG. 1 is a partial structural diagram of a surgical robot provided in an embodiment of the present application;

FIG. 2 is a Wheatstone bridge circuit in a strain gauge provided in an embodiment of the present application;

FIG. 3 is a demonstration diagram of installing a strain gauge on a mechanical arm provided in an embodiment of the present application;

FIG. 4 is a schematic diagram of a joint bending angle provided in an embodiment of the present application; and

FIG. 5 is a flow diagram of a positioning method for a surgical robot provided in an embodiment of the present application.

### DETAILED DESCRIPTION

The technical solutions in the embodiments of the present application will be described clearly and completely below in combination with the accompanying drawings in the embodiments of the present application. Obviously, the described embodiments are part of the embodiments of the present application, not all of them. Based on the embodiments in the present application, all other embodiments obtained by those skilled in the art without creative work belong to the scope of protection in the present application.

The terms “first”, “second”, “third” and “fourth” in the description, claims and accompanying drawings of the present application are used to distinguish different objects, not to describe a specific order. Furthermore, the terms “including” and “having” and any deformation thereof are intended to cover non-exclusive inclusion. For example, a process, method, system, product or equipment that includes a series of steps or units is not limited to the listed steps or units, but optionally also includes steps or units that are not listed, or optionally also includes other steps or units that are inherent to these processes, methods, products or equipment.

The reference herein to “embodiment” means that specific features, structures or characteristics described in connection with embodiments may be included in at least one embodiment of the present application. The phrase “embodiment” shown in various positions in the description does not necessarily refer to the same embodiment, nor an independent or alternative embodiment mutually exclusive with other embodiments. Those skilled in the art explicitly and implicitly understand that the embodiments described herein can be combined with other embodiments.

The technical solutions in the embodiments of the present application will be described in detail below in combination with the accompanying drawings in the embodiments of the present application.

### Embodiment One

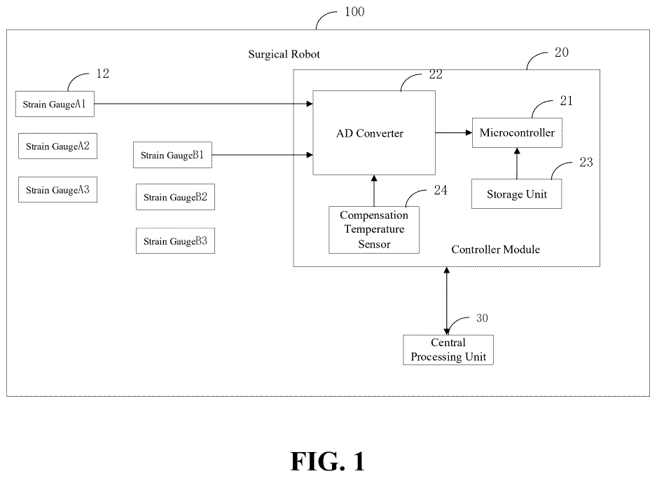

As shown in FIGS. 1 to 4, an embodiment of the present application proposes a surgical robot. FIG. 1 is a partial structural diagram of a surgical robot provided in the embodiment of the present application. The surgical robot 100 includes an operating arm 10. The operation arm includes a plurality of joint modules 11, the joint modules are connected with each other by joints, and a strain gauge 12 is provide at each joint connected between the joint modules respectively. The operation arm is connected with a central processing unit 30.

The operating arm 10 of the surgical robot 100 is composed of multiple hinged joint modules 11. The strain gauge is provided at each joint between the joint modules to sense a angle change of the joint. Through the central processing unit, the operation of the operating arm can be controlled according to the angle changes sensed by the strain gauge to achieve a closed-loop accurate control of the surgical robot.

The operating arm is provided with a controller module 20, which is connected with the strain gauge 12. The controller module includes a microcontroller 21, a AD converter 22 and a storage unit 23.

The AD converter is configured to perform analog-to-digital conversion on the data detected by the strain gauge. The storage unit is configured to store the data detected by the strain gauge. The microcontroller is configured to encode the data detected by the strain gauge, and send the encoded data to the central processing unit through a communication link.

The operating arm is detachably provided on the surgical robot. As a detachable basic assembly, one end of the operating arm may be provided with a controller module, which may include a microcontroller 21, a AD converter 22 and a storage unit 23. The strain gauge of each joint correspondingly transmits the detected data to the AD converter, and the AD converter transmits the converted data to the microcontroller. The converted data is encoded by the microcontroller, and the microcontroller transmits the encoded data to the central processing unit. Optionally, the controller module may also include a compensation temperature sensor 24 for performing thermal drift compensation, to reduce the impact of temperature noise on the detection results, so as to improve the accuracy of the detection results.

As shown in FIG. 3, more than three strain gauges may be installed on each joint, which can more accurately detect the angle bending change of the joint.

The surgical robot further includes a motor for driving the operation arm 10.

The mechanical arm 10 may also be controlled by an external system.

The central processing unit 30 is configured to:
obtain a joint bending angle of each joint in the present detection, and get first detected data of the joint bending angle corresponding to each joint, where the joint bending angle is determined according to a resistance parameter detected by the strain gauge corresponding to the joint;
get present first estimated data of the joint bending angle of each joint, according to previous second adjustment data of the joint angle of each joint.
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

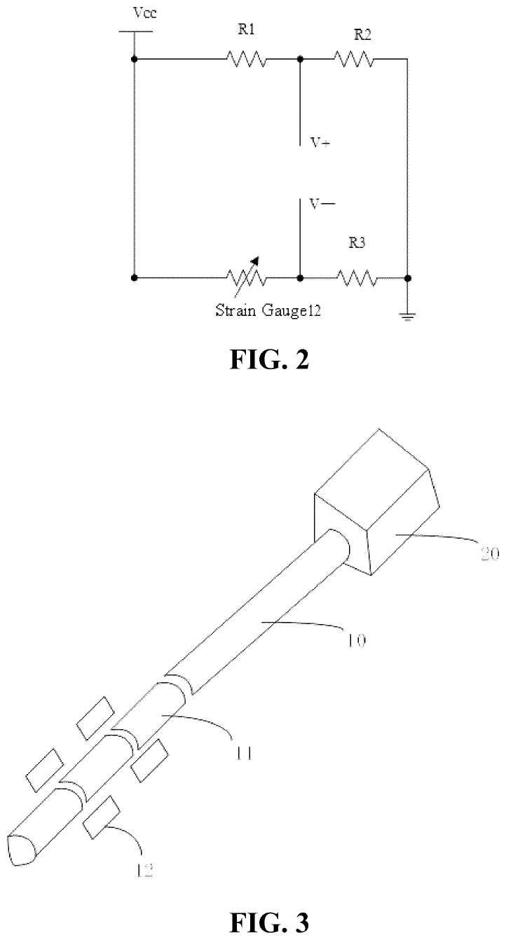

The strain gauge is a very small plate, which is composed of multiple resistors to form a bridge circuit, as shown in FIG. 2, which is a Wheatstone bridge circuit in the strain gauge provided in the embodiment of the present application. When the strain gauge bends or elongates, a resistance between the resistance bridge legs will change. The ratio of resistance difference is usually linearly related to the bending angle or elongation. The Wheatstone bridge with optional amplifier is used to enlarge and measure the resistance difference in the bridge, and the AD converter is used to digitize, so that the bending angle data of the joint can be detected.

The second adjustment data refers to the adjustment data of the previous joint angle of each joint, and the movement parameter could be the translation parameter of the tip. The adjustment data of the joint angle may be determined by combining the detected data and estimated data of the joint bending angle, and the present first adjustment data of the joint angle corresponding to the strain gauge may be determined according to the present first detected data of the joint bending angle and the present first estimated data of the joint bending angle corresponding to each joint, so as to calibrate the detected data of the joint bending angle and ensure the accuracy of the positioning of the orientation parameter and the movement parameter of the mechanical arm.

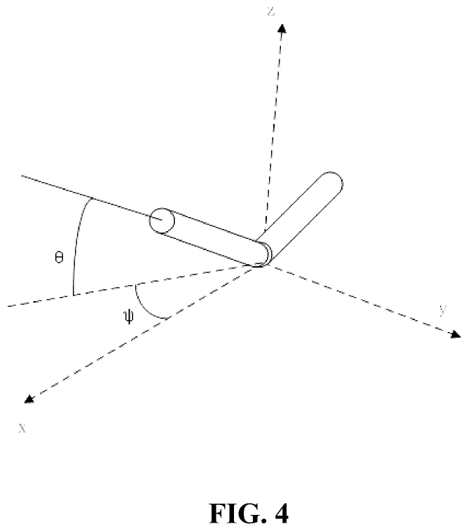

Optionally, more than two small strain gauges may be installed on each joint of the surgical robot. As shown in FIG. 3 to FIG. 4, two joint bending angles θ and y in a transverse plane may be calculated by installing more than three small strain gauges on each joint of the surgical robot. The pitch angle θ is an included angle between an axial vector and the XY plane. The yaw angle ψ is an included angle between the axial vector and the XZ plane.

Optionally, in terms of getting the present first estimated data of the joint bending angle of each joint, according to the previous second adjustment data of the joint angle of each joint, the central processing unit is specifically configured to:
obtain motor input angle data of the motor; and
determine the first estimated data of the joint bending angle corresponding to each joint according to the motor input angle data, the previous second adjustment data of the joint angle corresponding to each joint and a preset calibration parameter, and get the present first estimated data of the joint bending angle corresponding to each joint, where the preset calibration parameter comprises a kinematic parameter and a motor shaft parameter.

The motor input angle data is an angle of an encoder of the motor. For a single joint, the bending angle is linearly related to the angle of the encoder of the motor. Therefore, the first estimated data of the joint bending angle may be calculated from the angle of the encoder of the motor using a preset calibration parameter. All preset calibration parameters and variables are calibrated and monitored by the central processing unit, so the best estimates of the two joint bending angles can be found.

The kinematics parameter and the motor shaft parameter are parameters of motor itself, which are constants that do not change with time.

Optionally, in terms of determining the present first adjustment data of the joint angle corresponding to each joint, according to the present first detected data of the joint bending angle and the present first estimated data of the joint bending angle of each joint, the central processing unit is specifically configured to:
calculate bending angle covariance according to the present first estimated data of the joint bending angle of each joint;
obtain measured noise covariance of the strain gauge;
determine the Kalman gain according to the bending angle covariance, the measured noise covariance and a preset strain gauge parameter; and
determine the first adjustment data of the joint angle corresponding to each joint according to the present first estimated data of the joint bending angle, the present first detected data of the joint bending angle and the Kalman gain of each joint, and get the first adjustment data of the joint angle corresponding to each joint.

Optionally, in terms of obtaining an orientation parameter and a movement parameter of a tip of the operating arm, according to the first adjustment data of the joint angle corresponding to each joint and a length of the operating arm, the central processing unit is specifically configured to:
determine a spatial transformation matrix of each pairwise adjacent joint modules from the joint module at a position of the tip to the distal joint module until the proximal joint module according to the first adjustment data of the joint angle corresponding to each joint and the length of the operating arm, and get the spatial transformation matrix corresponding to each joint; and
determine the orientation parameter and the movement parameter of the tip according to the spatial transformation matrix corresponding to each joint.

The mechanical arm is composed of multiple hinged joint modules. Once the two joint angles of each hinged joint are adjusted and in place, the kinematic chain and the length of the joint may be used to calculate the orientation parameter and the movement parameter of the tip of the mechanical arm.

The surgical robot of the present application obtains the joint bending angle of each joint in the present detection, and gets the first detected data of the joint bending angle corresponding to each joint. The joint bending angle is determined according to the resistance parameter detected by the strain gauge corresponding to the joint. The present first estimated data of the joint bending angle of each joint is got according to the previous second adjustment data of the joint angle of each joint. The present first adjustment data of the joint angle corresponding to each joint is determined according to the present first detected data of the joint bending angle and the present first estimated data of the joint bending angle of each joint. The orientation parameter and the movement parameter of the tip of the operating arm is obtained according to the first adjustment data of the joint angle corresponding to each joint and the length of the operating arm. In this way, the adjustment data of the joint angle can be determined by combining the detected data and estimated data of the joint bending angle, and the detected data of the joint bending angle can be calibrated to reduce the impact of noise such as temperature on the detection results, so as to ensure the accuracy of positioning the position and direction of the tip of the operating arm.

### Embodiment Two

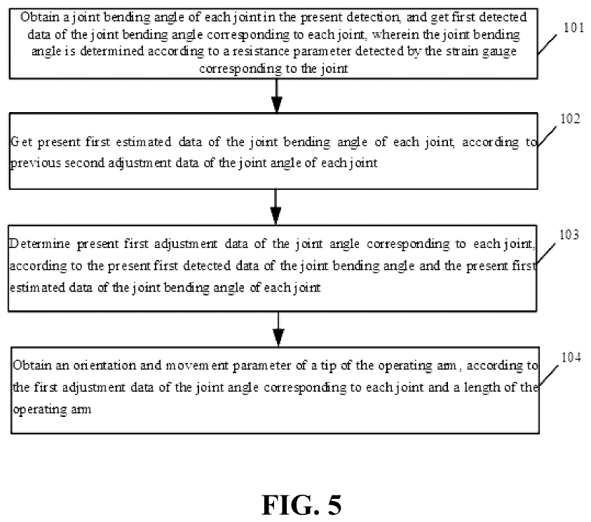

As shown in FIG. 5, FIG. 5 is a flow diagram of a positioning method for a surgical robot provided in an embodiment of the present application. An embodiment of the present application proposes a positioning method for a surgical robot, applied to a surgical robot, the surgical robot including an operating arm, the operating arm including a plurality of joint modules connected with each other, and a strain gauge is provide at each joint connected between the joint modules respectively, the method including the following steps.

At step 101: first detected data of the joint bending angle corresponding to each joint is got by obtaining a joint bending angle of each joint in the present detection, where the joint bending angle is determined according to a resistance parameter detected by the strain gauge corresponding to the joint.

When the joint connected between each joint module of the mechanical arm is bent or elongated, the bridge circuit provided on the strain gauge at the joint may detect the resistance parameter, and then determine the bending angle data of the joint. Therefore, the first detected data of the joint bending angle of each joint may be obtained. For example, three strain gauges are installed on one joint, and two bending angles (the pitch angle θ and the yaw angle ψ) corresponding to the joint may be determined, thus, the first detected data of the joint bending angle corresponding to each joint may be got.

At step 102: present first estimated data of the joint bending angle of each joint is got according to previous second adjustment data of the joint angle of each joint.

The first estimated data of the joint bending angle may be estimated by combining the second adjustment data of the joint angle of a previous period.

At step 103: present first adjustment data of the joint angle corresponding to each joint is determined, according to the present first detected data of the joint bending angle and the present first estimated data of the joint bending angle of each joint.

Specifically, by determining the present first adjustment data of the joint angle based on the first detected data of the joint bending angle and the first estimated data of the joint bending angle, the first detected data of the joint bending angle may be calibrated to improve the accuracy of the positioning of the mechanical arm.

At step 104: an orientation parameter and a movement parameter of a tip of the operating arm is obtained, according to the first adjustment data of the joint angle corresponding to each joint and a length of the operating arm.

The mechanical arm is composed of multiple hinged joint modules. Once the two joint angles at each hinged joint are adjusted and in place, the position and direction of the tip of the mechanical arm can be calculated by using the kinematic chain and the length of the joint.

In the positioning method for a surgical robot in the present application, the first detected data of the joint bending angle corresponding to each joint is got by obtaining the joint bending angle of each joint in the present detection. The joint bending angle is determined according to the resistance parameter detected by the strain gauge corresponding to the joint. The present first estimated data of the joint bending angle of each joint is got according to the previous second adjustment data of the joint angle of each joint. The present first adjustment data of the joint angle corresponding to each joint is determined according to the present first detected data of the joint bending angle and the present first estimated data of the joint bending angle of each joint. The orientation parameter and the movement parameter of the tip of the operating arm is obtained according to the first adjustment data of the joint angle corresponding to each joint and the length of the operating arm. In this way, the adjustment data of the joint angle can be determined by combining the detected data and estimated data of the joint bending angle, and the detected data of the joint bending angle can be calibrated to reduce the impact of noise such as temperature on the detection results, so as to ensure the accuracy of positioning the position and direction of the tip of the operating arm.

### Embodiment Three

In order to accurately control the mechanical arm of the surgical robot, the detected data may be fused with the Kalman filter, the extended Kalman filter or the unscented Kalman filter, and the linear quadratic equation (LQE) algorithm is used to calculate the first adjustment data of the joint angle corresponding to each joint, so as to more accurately determine the orientation parameter and the movement parameter of the tip of the operating arm, and achieve the precise positioning and precise control of the surgical robot.

The second embodiment of the present application also proposes a positioning method for a surgical robot. The method of the second embodiment is a further improvement of the method of the first embodiment. The main improvement is in that, in the second embodiment of the present application, at least two strain gauges are installed on each joint. The obtaining the joint bending angle of each joint in the present detection, and getting the first detected data of the joint bending angle corresponding to each joint includes:
obtaining the resistance parameter of each strain gauge corresponding to each joint, and getting first resistance parameter data; and
determining a joint bending yaw angle and a joint bending pitch angle corresponding to each joint according to the first resistance parameter data, and taking the joint bending yaw angle and the joint bending pitch angle corresponding to each joint as the first detected data of the joint bending angle corresponding to the joint.

As shown in FIG. 4, in the specific implementation, the two joint bending angles θ and w in the transverse plane may be calculated. The pitch angle θ is an included angle between the axial vector and the XY plane. The yaw angle ψ is an included angle between the axial vector and the XZ plane. By determining two bending angles (the pitch angle θ and the yaw angle ψ) corresponding to the joint, thus, the first detected data of the joint bending angle corresponding to each joint may be obtained.

Optionally, the surgical robot also includes a motor for driving the operating arm. The getting the present first estimated data of the joint bending angle of each joint, according to the previous second adjustment data of the joint angle of each joint, includes:
obtaining motor input angle data of the motor; and
determining the first estimated data of the joint bending angle corresponding to each joint according to the motor input angle data, the previous second adjustment data of the joint angle corresponding to each joint and a preset calibration parameter, and getting the present first estimated data of the joint bending angle corresponding to each joint, where the preset calibration parameter comprises a kinematic parameter and a motor shaft parameter.

In specific implementation, the mechanical arm may be driven by a motor, and may obtain the angle of the encoder of the motor, and get the motor input angle data. For a single joint, the bending angle is linearly related to the angle of the encoder of the motor. Therefore, the first estimated data of the joint bending angle may be calculated from the angle of the encoder of the motor using a preset calibration parameter. All preset calibration parameter and variable are calibrated and monitored by the central processing unit, so the best estimates of the two joint bending angles can be found. The calculation equation of the first estimated data of the joint bending angle is as follows:

$${X}_{n \vert n - 1} {AX}_{n - 1 \vert n - 1} + {BU}_{n} \qquad Equation 1$$

X(n|n−1) is the present (time n) estimated value of the bending angle corresponding to the joint. A and B are the kinematic parameter and the motor shaft parameter of the surgical robot, which are constants that do not change with time. Un is the present n time motor input angle.

Optionally, the determining the present first adjustment data of the joint angle corresponding to each joint, according to the present first detected data of the joint bending angle and the present first estimated data of the joint bending angle of each joint, includes:
calculating bending angle covariance according to the present first estimated data of the joint bending angle of each joint;
obtaining measured noise covariance of the strain gauge;
determining the Kalman gain according to the bending angle covariance, the measured noise covariance and a preset strain gauge parameter; and
determining the first adjustment data of the joint angle corresponding to each joint according to the present first estimated data of the joint bending angle, the present first detected data of the joint bending angle and the Kalman gain of each joint, and getting the first adjustment data of the joint angle corresponding to each joint.

In specific implementation, the equation for calculating the Kalman gain according to the bending angle covariance, the measured noise covariance and the preset strain gauge parameter is as follows:

$${K}_{n} = {P}_{n \vert n - 1} {H}^{T} ( {HP}_{n \vert n - 1} {H}^{T} + {R}_{k} ) \qquad Equation 2$$

Kn is the Kalman gain of the Kalman filter, extended Kalman filter or unscented Kalman filter. P_(n|n−1) is the covariance of the present (time n) estimated value of the bending angle corresponding to the joint. Rk is the covariance of the strain gauge measured noise. H is the strain gauge parameter, which is a constant that does not change with time.

The equation for determining the first adjustment data of the joint angle corresponding to each joint according to the first estimated data of the joint bending angle, the first detected data of the joint bending angle and the Kalman gain is as follows:

$${X}_{n \vert n} = {X}_{n \vert n - 1} + {K}_{n} ( {Z}_{n} - {HX}_{n \vert n - 1} ) \qquad Equation 3$$

X(n|n) is the present (time n) bending angle adjusted value corresponding to the joint. Zn is a detected value of two joint bending angles of the strain gauge at present (time n).

Optionally, the obtaining an orientation parameter and a movement parameter of a tip of the operating arm, according to the first adjustment data of the joint angle corresponding to each joint and a length of the operating arm, includes:
determining a spatial transformation matrix of each pairwise adjacent joint modules from the joint module at a position of the tip to the distal joint module until the proximal joint module according to the first adjustment data of the joint angle corresponding to each joint and the length of the operating arm, and getting the spatial transformation matrix corresponding to each joint; and
determining the orientation parameter and the movement parameter of the tip according to the spatial transformation matrix corresponding to each joint.

The mechanical arm is composed of multiple hinged joint modules. Once the two joint angles of each hinged joint are adjusted and in place, the position and direction of the tip of the mechanical arm can be calculated by using the kinematic chain and the length of the joint.

For example, the position of the tip, joint module 1 and joint module 2 are represented by c, b and a respectively. Sab is a spatial transformation matrix of joints from a distal joint module b to a proximal joint module a,

$${S}_{ab} = \begin{matrix}{R}_{11} & {R}_{12} & {R}_{13} & {T}_{x} \\ {R}_{21} & {R}_{22} & {R}_{23} & {T}_{y} \\ {R}_{31} & {R}_{32} & {R}_{33} & {T}_{z} \\ 0 & 0 & 0 & 1\end{matrix}$$

Rxx is the element of the rotation matrix from b to a. T is the translation vector from the joint b to the joint a. If the mechanical arm is a rigid link, the translation vector is a constant parameter, and the ending 6-dof information of c in the platform coordinate system is equal to SabSbcSc. Sc is the rotation vector of the position of the tip, which literally means Sc.

$${S}_{c} = \begin{matrix}{R}_{11} & {R}_{12} & {R}_{13} & 0 \\ {R}_{21} & {R}_{22} & {R}_{23} & 0 \\ {R}_{31} & {R}_{32} & {R}_{33} & 0 \\ 0 & 0 & 0 & 1\end{matrix}$$

In the positioning method for a surgical robot in the present application, the bending angle covariance is calculated according to the present first estimated data of the joint bending angle of each joint. The measured noise covariance of the strain gauge is obtained. The Kalman gain is determined according to the bending angle covariance, the measured noise covariance and the preset strain gauge parameter. The first adjustment data of the joint angle corresponding to each joint is determined according to the present first estimated data of the joint bending angle, the present first detected data of the joint bending angle and the Kalman gain of each joint, and the first adjustment data of the joint angle corresponding to each joint is got. The first adjustment data of the joint angle corresponding to each joint is calculated using the linear quadratic equation (LQE) algorithm, in order to more accurately determine the position and direction of the tip of the operating arm, to reduce the impact of temperature and other noises on the detection results, so as to achieve accurate positioning and precise control of the surgical robot.

### Embodiment Four

The fourth embodiment of the present application provides a positioning system for a surgical robot. The fourth embodiment is consistent with the system of the first embodiment and the method of the second embodiment. Specifically, in the fourth embodiment, the positioning system for a surgical robot is applied to a surgical robot, and the positioning system for a surgical robot includes an operating arm. The operation arm comprises a plurality of joint modules connected with each other, and a strain gauge is provide at each joint connected between the joint modules respectively. The operation arm is connected with a central processing unit, and the central processing unit is configured to:
obtain a joint bending angle of each joint in the present detection, and get first detected data of the joint bending angle corresponding to each joint, where the joint bending angle is determined according to a resistance parameter detected by the strain gauge corresponding to the joint;
get present first estimated data of the joint bending angle of each joint, according to previous second adjustment data of the joint angle of each joint;
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

Optionally, the operating arm is provided with a controller module, the controller module is connected with the strain gauge, and the controller module comprises a microcontroller, a AD converter and a storage unit, wherein,
the AD converter is configured to perform analog-to-digital conversion on the data detected by the strain gauge;
the storage unit is configured to store the data detected by the strain gauge; and
the microcontroller is configured to encode the data detected by the strain gauge, and send the encoded data to the central processing unit through a communication link.

Optionally, the surgical robot further includes a motor, which is configured to drive the operating arm. In terms of getting the present first estimated data of the joint bending angle of each joint, according to the previous second adjustment data of the joint angle of each joint, the central processing unit is specifically configured to:
obtain motor input angle data of the motor; and
determine the first estimated data of the joint bending angle corresponding to each joint according to the motor input angle data, the previous second adjustment data of the joint angle corresponding to each joint and a preset calibration parameter, and getting the present first estimated data of the joint bending angle corresponding to each joint, where the preset calibration parameter comprises a kinematic parameter and a motor shaft parameter.

Optionally, in terms of determining the present first adjustment data of the joint angle corresponding to each joint, according to the present first detected data of the joint bending angle and the present first estimated data of the joint bending angle of each joint, the central processing unit is specifically configured to:
calculate bending angle covariance according to the present first estimated data of the joint bending angle of each joint;
obtaining measured noise covariance of the strain gauge;
determining the Kalman gain according to the bending angle covariance, the measured noise covariance and a preset strain gauge parameter; and
determining the first adjustment data of the joint angle corresponding to each joint according to the present first estimated data of the joint bending angle, the present first detected data of the joint bending angle and the Kalman gain of each joint, and getting the first adjustment data of the joint angle corresponding to each joint.

Optionally, in terms of obtaining an orientation parameter and a movement parameter of a tip of the operating arm, according to the first adjustment data of the joint angle corresponding to each joint and a length of the operating arm, the central processing unit is specifically configured to:
determine a spatial transformation matrix of each pairwise adjacent joint modules from the joint module at a position of the tip to the distal joint module until the proximal joint module according to the first adjustment data of the joint angle corresponding to each joint and the length of the operating arm, and get the spatial transformation matrix corresponding to each joint; and
determining the orientation parameter and the movement parameter of the tip according to the spatial transformation matrix corresponding to each joint.

The specific structure and specific operation of the positioning system for a surgical robot are consistent with the specific implementation steps of the surgical robot and the positioning method mentioned above, and will not be repeated here.

In the positioning system for a surgical robot in the present application, the first detected data of the joint bending angle corresponding to each joint is got by obtaining the joint bending angle of each joint in the present detection. The joint bending angle is determined according to the resistance parameter detected by the strain gauge corresponding to the joint. The present first estimated data of the joint bending angle of each joint is got according to the previous second adjustment data of the joint angle of each joint. The present first adjustment data of the joint angle corresponding to each joint is determined according to the present first detected data of the joint bending angle and the present first estimated data of the joint bending angle of each joint. The orientation parameter and the movement parameter of the tip of the operating arm is obtained according to the first adjustment data of the joint angle corresponding to each joint and the length of the operating arm. In this way, the adjustment data of the joint angle can be determined by combining the detected data and estimated data of the joint bending angle, and the detected data of the joint bending angle can be calibrated to ensure the accuracy of positioning the position and direction of the tip of the operating arm.

The embodiments of the application also provide a computer-readable storage medium, which stores program instructions. When the program instructions are performed by a computer, cause the computer to perform the positioning method for a surgical robot as described above.

The embodiment of the application also provides a computer program product, wherein the computer program product includes a non-transitory computer-readable storage medium that stores computer programs, and the computer program is operable to enable the computer to perform some or all of the steps described in any positioning method of a surgical robot recorded in the embodiments of the present application. The computer program product can be a software installation package.

Although the present application is described herein in combination with various embodiments, in the process of implementing the claimed application, those skilled in the art can understand and realize other changes of the disclosed embodiments by viewing the accompanying drawings, the disclosure, and the appended claims. In the claims, the word “comprising” does not exclude other components or steps, and the word “a” or “one” does not exclude the case of multiple. A single processor or other unit may realize several functions listed in the claims. Some measures are recorded in different dependent claims, but this does not mean that these measures cannot be combined to produce good effects.

Those skilled in the art should understand that embodiments of the present application can be provided as a method, device (equipment), or computer program product. Therefore, the present application may take the form of an embodiment that uses entirely hardware, an embodiment that uses entirely software, or an embodiment combining software and hardware. Moreover, the present application may take the form of a computer program product implemented on one or more computer available storage media (including but not limited to a disk memory, CD-ROM, optical memory, etc.) containing computer available program codes. Computer programs are stored/distributed in appropriate media, provided together with other hardware or as part of the hardware, or in other distribution forms, such as through the Internet or other wired or wireless telecommunications systems.

The present application is described with reference to the flow chart and/or block diagram of the method, device (equipment) and computer program product of the embodiments of the present application. It should be understood that each flow and/or block in the flow chart and/or block diagram and the combination of flows and/or blocks in the flow chart and/or block diagram can be realized by computer program instructions. These computer program instructions can be provided to a general purpose computer, a special purpose computer, an embedded processor or a processor of other programmable human vehicle trajectory analysis equipment to generate a machine, so that the instructions performed by a computer or a processor of other programmable human vehicle trajectory analysis equipment can generate a device for realizing the functions specified in one or more flows in the flow chart and/or one or more blocks in the block diagram.

These computer program instructions may also be stored in a computer-readable memory that can guide a computer or other programmable human vehicle trajectory analysis equipment to work in a specific way, so that the instructions stored in the computer-readable memory generate a manufacture including an instruction device that realizes the functions specified in one or more flows in the flow chart and/or one or more blocks in the block diagram.

These computer program instructions can also be loaded onto a computer or other programmable human vehicle trajectory analysis equipment, so that a series of operation steps can be performed on the computer or other programmable device to generate a computer implemented process, so that the instructions performed on the computer or other programmable device provide steps for realizing the functions specified in one or more flows in the flow chart and/or one or more blocks in the block diagram.

Although the present application is described in combination with specific features and embodiments thereof, it is obvious that various modifications and combinations can be made without departing from the spirit and scope of the present application. Accordingly, the description and the accompanying drawings are only exemplary descriptions of the present application as defined in the appended claims, and are deemed to have covered any and all modifications, variations, combinations or equivalents within the scope of the present application. Obviously, those skilled in the art can make various changes and variations to the present application without departing from the spirit and scope of the present application. In this way, if these modifications and variations of the present application fall within the scope of the claims of the present application and their equivalent technologies, the present application is also intended to include these modifications and variations.

1. A positioning method for a surgical robot, applied to a surgical robot, the surgical robot comprising an operating arm, the operating arm comprising a plurality of joint modules connected with each other, and a strain gauge is provide at each joint connected between the joint modules respectively, the method comprising:
obtaining a joint bending angle of each joint in the present detection, and getting first detected data of the joint bending angle corresponding to each joint, wherein the joint bending angle is determined according to a resistance parameter detected by the strain gauge corresponding to the joint;
getting present first estimated data of the joint bending angle of each joint, according to previous second adjustment data of the joint angle of each joint;
determining present first adjustment data of the joint angle corresponding to each joint, according to the present first detected data of the joint bending angle and the present first estimated data of the joint bending angle of each joint; and
obtaining an orientation parameter and a movement parameter of a tip of the operating arm, according to the first adjustment data of the joint angle corresponding to each joint and a length of the operating arm.

2. The positioning method for a surgical robot according to claim 1, wherein, at least two strain gauges are installed on each joint;
the obtaining the joint bending angle of each joint in the present detection, and getting the first detected data of the joint bending angle corresponding to each joint, comprises:
obtaining the resistance parameter of each strain gauge corresponding to each joint, and getting first resistance parameter data; and
determining a joint bending yaw angle and a joint bending pitch angle corresponding to each joint according to the first resistance parameter data, and taking the joint bending yaw angle and the joint bending pitch angle corresponding to each joint as the first detected data of the joint bending angle corresponding to the joint.

3. The positioning method for a surgical robot according to claim 1, wherein, the surgical robot further comprises a motor for driving the operating arm;
the getting the present first estimated data of the joint bending angle of each joint, according to the previous second adjustment data of the joint angle of each joint, comprises:
obtaining motor input angle data of the motor; and
determining the first estimated data of the joint bending angle corresponding to each joint according to the motor input angle data, the previous second adjustment data of the joint angle corresponding to each joint and a preset calibration parameter, and getting the present first estimated data of the joint bending angle corresponding to each joint, wherein the preset calibration parameter comprises a kinematic parameter and a motor shaft parameter.

4. The positioning method for a surgical robot according to claim 1, wherein, the determining the present first adjustment data of the joint angle corresponding to each joint, according to the present first detected data of the joint bending angle and the present first estimated data of the joint bending angle of each joint, comprises:
calculating bending angle covariance according to the present first estimated data of the joint bending angle of each joint;
obtaining measured noise covariance of the strain gauge;
determining the Kalman gain according to the bending angle covariance, the measured noise covariance and a preset strain gauge parameter; and
determining the first adjustment data of the joint angle corresponding to each joint according to the present first estimated data of the joint bending angle, the present first detected data of the joint bending angle and the Kalman gain of each joint, and getting the first adjustment data of the joint angle corresponding to each joint.

5. The positioning method for a surgical robot according to claim 1, wherein, the obtaining an orientation parameter and a movement parameter of a tip of the operating arm, according to the first adjustment data of the joint angle corresponding to each joint and a length of the operating arm, comprises:
determining a spatial transformation matrix of each pairwise adjacent joint modules from the joint module at a position of the tip to the distal joint module until the proximal joint module according to the first adjustment data of the joint angle corresponding to each joint and the length of the operating arm, and getting the spatial transformation matrix corresponding to each joint; and
determining the orientation parameter and the movement parameter of the tip according to the spatial transformation matrix corresponding to each joint.

6. A positioning system for a surgical robot, characterized in that, the positioning system for a surgical robot is applied to a surgical robot and comprises an operating arm; the operation arm comprises a plurality of joint modules connected with each other, and a strain gauge is provide at each joint connected between the joint modules respectively; and the operation arm is connected with a central processing unit, and the central processing unit is configured to:
obtain a joint bending angle of each joint in the present detection, and get first detected data of the joint bending angle corresponding to each joint, wherein the joint bending angle is determined according to a resistance parameter detected by the strain gauge corresponding to the joint;
get present first estimated data of the joint bending angle of each joint, according to previous second adjustment data of the joint angle of each joint;
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

7. The positioning system for a surgical robot according to claim 6, wherein, the operating arm is provided with a controller module, the controller module is connected with the strain gauge, and the controller module comprises a microcontroller, a AD converter and a storage unit, wherein,
the AD converter is configured to perform analog-to-digital conversion on the data detected by the strain gauge;
the storage unit is configured to store the data detected by the strain gauge; and
the microcontroller is configured to encode the data detected by the strain gauge, and send the encoded data to the central processing unit through a communication link.

8. The positioning system for a surgical robot according to claim 6, wherein, the surgical robot further comprises a motor, the motor is configured to drive the operating arm, and in terms of getting the present first estimated data of the joint bending angle of each joint, according to the previous second adjustment data of the joint angle of each joint, the central processing unit is specifically configured to:
obtain motor input angle data of the motor; and
determine the first estimated data of the joint bending angle corresponding to each joint according to the motor input angle data, the previous second adjustment data of the joint angle corresponding to each joint and a preset calibration parameter, and getting the present first estimated data of the joint bending angle corresponding to each joint, wherein the preset calibration parameter comprises a kinematic parameter and a motor shaft parameter.

9. A surgical robot, wherein, the surgical robot comprises an operating arm; the operation arm comprises a plurality of joint modules connected with each other, and a strain gauge is provide at each joint connected between the joint modules respectively; and the operation arm is connected with a central processing unit, and the central processing unit is configured to:
obtain a joint bending angle of each joint in the present detection, and getting first detected data of the joint bending angle corresponding to each joint, wherein the joint bending angle is determined according to a resistance parameter detected by the strain gauge corresponding to the joint;
get present first estimated data of the joint bending angle of each joint, according to previous second adjustment data of the joint angle of each joint;
determine present first adjustment data of the joint angle corresponding to each joint, according to the present first detected data of the joint bending angle and the present first estimated data of the joint bending angle of each joint; and
obtain an orientation parameter and a movement parameter of a tip of the operating arm, according to the first adjustment data of the joint angle corresponding to each joint and a length of the operating arm.

10. A computer-readable storage medium storing program instructions, wherein, when the program instructions are performed by a computer, cause the computer to perform the positioning method for a surgical robot according to claim 1.
